(19) United States
(12) Patent Application Publication
(10) Pub. No.: US 2023/0181941 A1
(43) Pub. Date: Jun. 15, 2023

(54) METHODS OF INACTIVATING MICROBIOLOGICAL CONTAMINATION

(71) Applicant: Osmotex AG, Thalwil (CH)

(72) Inventor: Trond Heldal, Luzern (CH)

(73) Assignee: Osmotex AG, Thalwil (CH)

(21) Appl. No.: 17/920,134

(22) PCT Filed: Apr. 21, 2021
(86) PCT No.: PCT/EP2021/060360

§ 371 Date: Oct. 20, 2022

(30) Foreign Application Priority Data
Apr. 21, 2020 (GB) 2005790.7
Jul. 8, 2020 (GB) 2010496.4

(51) Int. Cl. A62B 18/08; A61L 2/18; A62B 18/02; A41D 13/11; A41D 31/30

(52) U.S. Cl. CPC A62B 18/08; A41D 13/1192; A41D 31/30; A61L 2/183; A61L 2/186; A62B 18/02; A61L 2101/02; A61L 2202/11; A61L 2202/26

(57) ABSTRACT

Methods of inactivating microbiological contamination described herein use a textile or membrane which can generate a contamination-inactivating amount of ozone or a reactive oxygen species. The textile or membrane includes first and second conductive layers and at least one ion conductive or porous intermediate layer positioned between the first and second conductive layers. The textile or membrane can form part of a protective face mask, for example a medical or surgical face mask. A voltage effective to generate a microbiological contamination-inactivating amount of the inactivating species is applied across the intermediate layer of the textile or membrane.

This application claims the priority of International Application PCT/EP2021/060360, filed Apr. 21, 2021, and GB 2005790.7, filed Apr. 21, 2020, and GB 2010496.4, filed Jul. 8, 2020, from which the PCT Application claims priority, the disclosures of which are incorporated herein by reference in their entireties.

### FIELD

The invention relates to methods of inactivating microbiological contamination using a textile or membrane which can generate a contamination-inactivating amount of ozone or a reactive oxygen species.

### BACKGROUND TO THE INVENTION

One of the major routes of contagion for bacteria and viruses, including SARS CoV-2, the infectious agent for COVID-19, is via surfaces in public areas, offices or hospitals, on which viruses can survive for weeks. Furthermore, bacteria, viruses and other contamination can adhere to garments, gloves and face masks, which may be of significance in controlling hospital infection.

Many other physical surfaces are, or can be, covered by textile materials, including seating and interior panels in offices or public transport or light walls and delimiters in offices.

The present invention concerns an electronic disinfection textile or membrane material which can potentially be used on most types of surfaces and can be incorporated into garments, gloves and face masks.

Electro-osmotic materials are known for their liquid transport properties, see for example WO 1999/000166 which describes a structure of three or more layers in which a conductor or semi-conductor is laminated to each side of a porous or textile intermediate layer. An applied voltage causes liquid to migrate through the material.

Further developments of this original concept can be seen, for example, in WO 2009/024779 and in WO 2019/053064, which discloses some of the electrolytic processes taking place when lower voltages are applied to the material. However, none of these documents suggests a sterilising or decontaminating function for these materials.

Ozone and hydrogen peroxide are widely used for sterilization, for instance in water purification. While toxic in higher concentrations, both of these agents are used in medicine, including for their antiviral and antibacterial effect as well as other beneficial effects, e.g., to the human skin. An advantage of ozone and hydrogen peroxide is that they break down to oxygen and water after short time. Both disinfecting agents are in broad industrial use, and there is a significant volume of published research on their effects. For example, a strong reduction in the presence of 12 different viruses (three orders of magnitude reduction in concentration) has been demonstrated, using a concentration of 20-25 ppm of ozone at high air humidity (>90%), see Hudson JB, Sharma M, Vimalanathan S, Development of a Practical Method for Using Ozone Gas as a Virus Decontaminating Agent in Ozone: Science & Engineering, vol. 31, p. 216-223 (2009).

Ozone concentrations of 0.5-2 ppm have been reported to be sufficient for “purification or ultra-purification of water for different purposes (e.g., pharmaceutical and electronic industries, water bottling process, etc.)” (see Da Silva LM, Franco DV, Goncalves IC, Sousa LG (2009) In: Gertsen N, Sonderby L (eds) Water purification. Nova Science Publishers Inc., New York; and Tchobanoglous G, Burton FL, Stensel HD (2003) Wastewater engineering: treatment and reuse, 4^{th} edn. Metcalf & Eddy Inc., New York). See also De Sousa et al. in J. Environmental Chem. Eng. 4 (2016), pages 418-427 for an electrochemical ozone generator.

### SUMMARY OF THE INVENTION

According to one embodiment the invention provides a method of inactivating microbiological contamination at a locus, the locus including a textile or membrane including first and second conductive layers and at least one ion conductive or porous intermediate layer positioned between the first and second conductive layers. The textile or membrane further includes an aqueous liquid on a surface of the textile or membrane or in the pores of a porous intermediate layer. The method includes applying, across the intermediate layer of the textile or membrane, a voltage effective to generate a microbiological contamination-inactivating amount of an inactivating species selected from ozone and reactive oxygen species.

A further embodiment of the invention provides a method of inactivating microbiological contamination at a locus. The method includes contacting the locus with a textile or membrane including first and second conductive layers and at least one ion conductive or porous intermediate layer positioned between the first and second conductive layers. The textile or membrane further includes an aqueous liquid on a surface of the textile or membrane or in the pores of a porous intermediate layer. The surface of the textile or membrane contacted with the locus includes a microbiological contamination-inactivating amount of an inactivating species selected from ozone and reactive oxygen species.

A yet further embodiment of the invention provides a protective face mask comprising a textile or membrane. The textile or membrane includes first and second conductive layers and at least one ion conductive or porous intermediate layer positioned between the first and second conductive layers. Preferably, the conductive layers are connected to an electric signal generator such that, in use, a voltage can be applied across the intermediate layer.

### DETAILED DESCRIPTION

The invention utilizes a flexible textile (i.e., woven material) or membrane (i.e., continuous material) comprising first and second conductive layers and at least one ion conductive or porous intermediate layer positioned between the first and second conductive layers. When the intermediate layer is wetted and a suitable voltage is applied across it, the textile or membrane generates a microbiological contamination-inactivating amount of an inactivating species selected from ozone and reactive oxygen species.

For application of a suitable voltage across the intermediate layer the conductive layers are connected to an electric signal generator, either in a fixed manner or temporarily.

The electrochemical generation of reactive oxygen species requires the presence of water or another aqueous liquid in the textile or membrane, either on the surface of the textile or membrane, or in the pores of a porous intermediate layer. The water or other liquid can be applied to the textile or membrane when required, for example by spraying from an external source, or it may be absorbed directly from the surrounding air if a more hygroscopic material has been incorporated into the intermediate layer. Depending on the use to which the textile or membrane is being put, the frequency of application of water or other liquid may need to be higher or lower, thus in certain applications the area being treated may require regular spraying so as to provide continuous inactivation of microbiological contamination. For example, spraying once per hour, twice per hour or three times per hour may be appropriate.

In other applications it may only be necessary to spray the textile or membrane with water, and apply a suitable voltage to generate the inactivating species, at less frequent intervals, such as once, twice or three times a day in connection with periodic cleaning of the potentially contaminated area.

In a yet further embodiment, for example a protective face mask to be worn by a user, the humidity generated by the breathing of the user may be sufficient to generate the necessary water. Under these circumstances, continuous inactivation of the microbiological contamination can be achieved by application of a continuous or suitably pulsed voltage across the intermediate layer.

The conductive layers in the textile or membrane utilised in the invention are typically selected from woven or non-woven conductive carbon, a textile layer comprising steel or silver or other metal yarn, metal layers, and graphene layers. The conductive layers will typically range from 50 to 500 micrometers in thickness.

The presence of metal ions released from the conductive layer may enhance the production of reactive oxygen species, for example via the Fenton Reaction illustrated in steps (1)-(3) below:

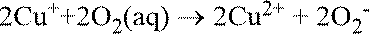

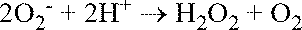

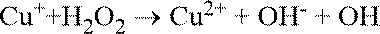

Therefore, conductive layers comprising metals such as Cu or Ag are also preferred in the textile or membrane according to the invention, to enhance the sterilizing effect of the material.

The intermediate layer is ion conductive or porous, in order for the applied voltage to generate an electric current via the ionic conductivity of the material or via an electrolytic mechanism as disclosed in, for example, WO 2019/053064 for lower voltages.

Suitable ion conductive materials include the sulfonated fluoropolymers which are, for example, commercially available from The Chemours Company under the name “Nafion”, i.e., tetrafluoroethylene-perfluoro-3,6-dioxa-4-methyl-7-octenesulfonic acid copolymers.

Alternative ion conductive materials are sulfonated “pentablock” copolymers with a t-butyl styrene, hydrogenated isoprene, sulfonated styrene, hydrogenated isoprene and t-butyl styrene (tBS-HI-SS-HI-tBS) structure, which are commercially available from Kraton Performance Polymers under the name “Nexar”.

A porous intermediate layer may comprise a water-swellable cross-linked polystyrene polymer, for example a styrene-divinylbenzene copolymer, or it may comprise polyethylene terephthalate. The term “porous” should be understood to cover so-called nanoporous materials with pore sizes in the range 0.1-1000 nm, microporous materials with pore sizes in the micrometre range (1-100 0micrometre ) as well as materials with pore-sizes up to a few (e.g., 3) mm. The important feature is the presence of voids (pores) large enough admit liquids, typically water. Preferably the average pore side is between 0.03 and 100 µm, and more preferably between 0.1 and 1 µm.

The intermediate layer may comprise a hygroscopic or water absorbing material, for example a cross linked hydrogel such as polyvinyl alcohol, sodium polyacrylate or other acrylate polymers, or the cross-linked polystyrene discussed above. In these embodiments, water with a significant concentration of ozone or reactive oxygen species will be present at the textile or membrane surface without the intermediate layer holding large amounts of water.

In particular, water can be held inside, or at the surface of, textile fibres as thin films, instead of the porous structure of the textile needing to be filled with water in order for wetting to be efficient.

Typically, the intermediate layer is between 2 µm and 1000 µm in thickness, preferably between 10 µm and 100 µm.

One particularly useful embodiment of the invention is a construction in which the textile or membrane forms a sterilisable cover for a face mask, for example a medical or surgical face mask. The face mask is thus potentially rendered reusable, or alternatively the lifetime of the face mask can be extended, as microbiological contamination can be inactivated with the cover in situ or the cover can be removed for separate treatment. The protection of the wearer will also be better, as compared to wearing a mask without the cover, because contamination accumulating in the mask and possibly being released by skin contact or breathing can be inactivated.

Other medical or surgical garments and personal protective equipment may likewise benefit from incorporation of a textile or membrane according to the present invention, or contact with a textile or membrane according to the invention, in order to inactivate microbiological contamination.

The invention will also find application in other areas requiring regular or occasional sterilisation or cleaning, so as to prevent infection where multiple users come into contact with an object or surface. Potential uses include garments and protective wear for use outside the medical or surgical environments described above. Additionally, utilization in transportation, in offices and in public places is envisaged: for example, a seat can include one or more arm rests which contain, or are cleaned with, a textile or membrane according to the invention.

Treatment of microbiological contamination on architectural features such as walls and light walls/delimiters, or on frequently used items such as tables, desks, door handles and the handles or surfaces of office equipment, is also included within the invention.

A portable pad or carpet made of the materials according to the invention can be carried by a user, for example on airplanes or public transport, in rental cars or ride-hailing vehicles, or at offices, and powered by a power bank or USB charger.

The microbiological contamination addressed by the invention can be bacterial contamination, or viral contamination, or any other form of contamination spread by airborne droplets, by contact or by other known routes. Of particular relevance at present is SARS CoV-2, the infectious agent for the disease COVID-19, but other contamination is addressed by the methods and articles according to the invention, such as influenza viruses, common cold viruses, mycobacteria (the causative agent of TB) and infectious fungi and spores.

The invention uses an inactivating species selected from ozone and reactive oxygen species, which it has unexpectedly been found can be produced in effective amounts when a suitable voltage is applied to the textile or membrane according to the invention. A voltage of 0.3 or 0.7 to 10.0 V is generally suitable, for example, to provide the desired current which may be either a continuous direct current or a pulsed direct current. Preferable the voltage is from 1.0 to 5.0 V and more preferably the voltage is from 1.0 to 3.0, in order to produce effective amounts of the inactivating species.

Alternatively, a low-frequency or long-period alternating current can have an amplitude or maximum voltage between 0.3 and 10.0 V, for example between 0.6 and 1.5 V, with a square pulse signal with signal period between 1 second and 100 minutes, preferably between 10 seconds and 10 minutes. Alternatively, the signal can have a sinusoidal or other shape and/or may include periods of zero voltage, for example of duration 5 minutes, spaced at regular intervals, for example every hour.

Reactive oxygen species are known and are generally regarded as including inter alia superoxide anions, hydrogen peroxide and hydroxyl radicals. Of these, hydrogen peroxide is the most commonly generated in the textile or membrane according to the invention and is the most useful in treating microbiological contamination. Suitable amounts of hydrogen peroxide are generally 1% to 90% by weight in aqueous solution, for example 1% to 5% or from 3% to 10% by weight in aqueous solution.

Furthermore, ozone can be generated in the textile or membrane according to the invention alongside, or instead of, the reactive oxygen species described above. A suitable concentration of ozone for inactivating microbiological contamination is 0.01 to 100 ppm by weight in water, for example 0.1 to 5.0 ppm, and/or 0.5 to 100 ppm by weight in air, for example 20 to 25 ppm.

Microbiological contamination coming into contact with the material of the invention will be inactivated by the generated ozone and/or reactive oxygen species. In order to enhance the effect, an antimicrobial coating can be included in, or coated onto, the electrically conductive textile or membrane of the invention. Examples include ion conductive and ion exchange compounds with fixed positive, or negative, or both positive and negative, charges. Especially, cationic species such as alkyl ammonium ions, cationic peptides, polymers with quaternary ammonium moieties such as chitosan or polymers with grafted positively charged groups can be effective. Optionally, such coatings can be mixed with a conductor such as graphene powder, other carbon or metal powder or fibres to maintain a high surface conductivity. Due to the electrical properties of such coating, synergistic effects with the electric field may be obtained, creating a stronger sterilizing effect than either the fabrics of the invention without such coatings, or the coatings when applied onto conventional materials such as standard textiles.

The electrochemical generation of ozone and hydrogen peroxide is known in the art, but is put to an unexpected use in the methods and articles of the present invention. The following examples of the invention illustrate this beneficial effect in a variety of conductive and intermediate layers.

### EXAMPLES

### 1. Ozone Generation

A series of tests were carried out with measurement of ozone as follows:

A three-layer fabric of area 80 cm² was wetted with between 1 and 5 ml water.

A voltage between 1.0 and 3.5 V was applied for 10 minutes.

The sample was put in water and subsequently a chemical analysis of the ozone content in the water sample was carried out.

In most tests, between 1 and 7 micrograms of ozone were detected, corresponding to concentrations between 0.1 and 4 ppm in the moisture contained in each sample.

By using a smaller water content in the sample (down to 250 microliter per 100 cm²) a maximum ozone concentration of 62 ppm could be obtained after 10 minutes of applying the voltage.

A concentration of 1 ppm is sufficient to kill most bacteria and viruses within 10 minutes.

The following abbreviations and/or materials are used in Table 1 below, reporting the results of the above tests:
sPET200™: porous polyethylene terephthalate membrane available from Osmotex AG, Switzerland
Steel mesh: from G Bopp & Co AG, Switzerland
Steel mesh TWP: from TWP Inc., USA
NuVant™: graphitized carbon from NuVant Systems Inc., USA
“carbon” - a carbon fabric from WidePlus International, Taiwan

TABLE 1

| Membrane | Electrodes (conductive layers) | Voltage Average [V] | Current [mA] | O₃ [mg/L] | O₃ mass [µg] |
|---|---|---|---|---|---|
| Nafion™ | steel mesh | 2.4 | 250 | 0.8 | 5.2 |
| 2x sPET200™ | steel mesh | 3.5 | 250 | 0.1 | 0.6 |
| sPET200™ | steel mesh | 1.0 | 1200 | 0.2 | 1.5 |
| sPET200™-reinforced with nonwoven | steel mesh | 2.0 | 500 | 0.1 | 1.1 |
| sPET200™ | steel mesh | 1.5 | 400 | 0.3 | 1.0 |
| sPET200™ | steel mesh | 1.2 | 400 | 0.2 | 1.5 |
| sPET200™ | carbon | 2.6 | 60 | 0.1 | 1.2 |
| sPET200™ | carbon | 1.7 | 20 | 0.2 | 2.1 |
| sPET200™ | steel mesh | 1.2 | 50 | 0.1 | 1.0 |
| sPET200™ | carbon | 2.1 | 60 | 0.1 | 0.8 |
| sPET200™ | carbon | 2.1 | 8 | 0.1 | 0.5 |
| sPET200™ | carbon | 1.4 | 30 | 0.5 | 4.1 |
| sPET200™ | carbon | 1.3 | 150 | 0.3 | 4.1 |
| sPET200™ | carbon | 1.0 | 4 | 0.2 | 2.8 |
| sPET200™ | carbon | 1.3 | 6 | 0.1 | 0.5 |
| Nafion™ | Steel mesh | 3.0 | 150 | 0.7 | 6.8 |
| Nafion™ | Steel mesh TWP | 3.0 | 30 | 0.2 | 1.3 |
| Nafion™ | Steel mesh | 3.0 | 140 | 0.5 | 4.2 |
| Nafion™ | Steel mesh TWP | 3.0 | 20 | 0.2 | 2.5 |
| sPET200™ | carbon | 1.3 | 40 | 0.2 | 2.0 |
| Nafion™ | Steel mesh | 3.0 | 20 | 0.3 | 2.1 |
| Nafion™ | Pt mesh (anode) NuVant™ carbon (cathode) | 3.0 | 210 | 0.5 | 0.9 |
| sPET200™ | carbon | 3.0 | 10 | 0.3 | 1.4 |
| Nafion™ | Steel mesh TWP | 5.0 | 100 | 2.2 | 4.5 |
| PET200™ | Au-coated steel | 2.9 | 200 | 3.8 | 2.7 |
| PET200™ | Au-coated steel | 1.7 | 1000 | 1.6 | 3.1 |
| Nafion™ | Steel mesh TWP | 5.0 | 120 | 7.1 | 10.1 |

### Inactivation Efficacy Against Bacteriophage MS2

Bacteriophage MS2 (a virus infecting the bacterium E. coli and belonging to the same taxonomic kingdom as coronaviruses) was used as a model organism to assess the ability of the membrane of the invention to inactivate viruses.

Based on the results of this experiment, a reduction of around 4 log levels (or 99.99% killing efficacy) was obtained for bacteriophage MS2, and an antiviral activity value (Mv) of 3.92 according to ISO 18184. An antiviral activity above 3 is rated as an “Excellent effect” in ISO 18184.

Note also that the log 4 reduction obtained above relates to the inactivation level of virus released from the fabric. Additionally taking into account the virus remaining in the fabric after the washing out step, i.e., virus particles which will have been highly exposed to the inactivating agents and most likely destroyed, the difference could be as high as 99.9999% or 6 log levels.

### Method

200 µl of bacteriophage MS2 suspension was applied to the pre-wetted membrane at a concentration of 10⁹ phage forming units (PFU) / ml and processed as shown in Table 2. As a control, the same amount of phage was applied to a second membrane and kept at room temperature without any treatment. A second control was included by adding 200 µl of the MS2 suspension to 20 ml of SCDLP medium to exclude effects due to medium compounds. In addition, the concentration of the initial MS2 phage suspension was verified.

TABLE 2

Treatment parameters

| | |
|---|---|
| Membrane used: | Nafion ™ ion conductive membrane with TWP steel mesh electrodes (conductive layers) |
| Membrane area: | 5 cm × 5 cm |
| Electrodes area: | 4.5 cm × 4.5 cm |
| Upward electrode (facing virus): | anode |
| Downward electrode: | cathode |
| Voltage applied: | 5 V |
| Power source: | XANTREX XKW ™ 150-7 |
| Treatment time: | 15 minutes |

### Recovery of Phages From Membrane

Phages were recovered from the membrane by cutting the membrane in two parts, placing them in 20 ml SCDLP-Medium (according to ISO 18184) in a 50 ml falcon tube and vortexing 5 times for 5 seconds at maximal speed.

### PFU Determination

A decimal dilution series in SCDLP-Medium was used to determine the number of surviving phages. Briefly, for each dilution, the 100 µl of a phage dilution was mixed with 3 ml of soft agar and 250 µl of 10⁷ colony forming units (CFU) / ml of the E. coli indicator strain and poured onto the surface of an agar plate (LB). After incubation at 37° C., the plaques were counted and the inactivation rates calculated. Results are shown in Table 3.

TABLE 3

Results

| Phage concentration applied to membrane: | 2.04 × 10⁹ PFU/ml |
|---|---|
| Phages recovered from control membrane (treatment control) | 9.73 × 10⁷ PFU/ml |
| Calculated recovery rate (=PFU applied/no treatment control) | 4.78% |
| Phages recovered from membrane after treatment (=PFU treated) | 1.18 × 10⁴ PFU/ml |
| Inactivation rate (PFU applied/PFU treated) | 0.012% |
| Percentage of inactivated phages | 99.99% |
| Antiviral activity value (Mv) according to ISO 18184 | 3.92 |

### Inactivation Efficacy Against Escherichia Coli Top10

Escherichia coli, a common human and animal pathogen, was used as a model organism to assess the potential of the membrane to inactivate bacteria. Based on the results of this experiment, a complete eradication was obtained for E.coli Top10 using the membrane of the invention.

### Method

200 µl of bacterial suspension of E.coli was applied to the pre-wetted membrane at a concentration of 1 × 10⁴ colony forming units (CFU) / ml and processed as shown in Table 4. As a control, the same amount of bacteria was applied to a second membrane and kept at room temperature without any treatment. In addition, the concentration of the initial bacterial suspension was verified.

TABLE 4

Treatment parameters

| | |
|---|---|
| Membrane used: | Nafion ™ ion conductive membrane with TWP steel mesh electrodes (conductive layers) |
| Membrane area: | 5 cm × 5 cm |
| Electrodes area: | 4.5 cm × 4.5 cm |
| Upward electrode (facing virus): | anode |
| Downward electrode: | cathode |
| Voltage applied: | 5 V |
| Power source: | XANTREX XKW™ 150-7 |
| Treatment time: | 15 minutes |

### Recovery of Bacteria From Membrane

Bacteria were recovered from the membrane by cutting the membrane in two parts, placing them in 20 ml SCDLP-Medium (according to ISO 18184) in a 50 ml falcon tube and vortexing 5 times for 5 seconds at maximal speed.

### CFU Determination

A decimal dilution series in physiological NaCl solution (0.9%) was used to determine the number of surviving bacteria. For each dilution 200 µl of bacterial suspension was plated on LB agar plates. The colonies were counted after 24 hours and the percentage of killed bacteria was calculated. Results are shown in TABLE 5.

TABLE 5

Results

| Number of bacteria applied to membrane: | 7.5 × 10³ CFU/ml |
|---|---|
| Bacteria recovered from control membrane (no treatment control) | 3.0 × 10² CFU/ml |
| Calculated recovery rate (=no treatment control / bacteria applied) | 4.0% |
| Bacteria recovered from membrane after treatment (=CFU treated) | 0 CFU/ml |
| Percentage of killed bacteria | 100% |

1. A method of inactivating microbiological contamination at a locus, said locus comprising a textile or membrane comprising first and second conductive layers and at least one ion conductive or porous intermediate layer positioned between said first and second conductive layers, the textile or membrane further comprising an aqueous liquid on a surface of said textile or membrane or in the pores of a porous intermediate layer;
wherein the method comprises applying across the intermediate layer of said textile or membrane a voltage effective to generate a microbiological contamination-inactivating amount of an inactivating species selected from ozone and reactive oxygen species.

2. (canceled)

3. The method of claim 1, wherein the microbiological contamination is bacterial contamination.

4. The method of claim 1, wherein the microbiological contamination is viral contamination.

5. The method of claim 4, wherein the viral contamination is SARS CoV-2.

6. The method of claim 1, wherein the conductive layers are selected from woven or non-woven conductive carbon, a textile comprising steel or silver yarn, metal layers, and graphene layers.

7. The method of claim 1, wherein the intermediate layer is a water-swellable cross-linked polystyrene polymer or a sulfonated fluoropolymer.

8. The method of claim 1, wherein said locus is a protective face mask.

9. The method of claim 8, wherein said face mask is a medical or surgical face mask.

10. The method of claim 8, wherein the textile or membrane is configured as a removable cover for said face mask.

11. The method of claim 1, wherein the applied voltage is from 0.3 to 10.0 V.

12. The method of claim 11, wherein the applied voltage is from 0.7 to 10.0 V.

13. The method of claim 12, wherein the applied voltage is from 1.0 to 5.0 V.

14. The method of claim 1, wherein an alternating voltage is applied across the intermediate layer.

15. The method of claim 14, wherein the alternating voltage has a waveform with a period between 10 seconds and 10 minutes and a maximum amplitude between 0.3 and 10.0 V.

16. The method of claim 1, wherein the reactive oxygen species is selected from ozone and hydrogen peroxide.

17. The method of claim 1, wherein one or more layers of said textile or membrane is coated with a coating comprising an ionic compound.

18. The method of claim 17, wherein the ionic compound is a polymer with fixed negative, fixed positive, or fixed negative and positive charges.

19. The method of claim 17, wherein the ionic compound is selected from the group comprising chitosan and peptides.

20. The method of claim 17, wherein said coating comprises electrically conductive particles.

21. A protective face mask comprising a textile or membrane, said textile or membrane comprising first and second conductive layers and at least one ion conductive or porous intermediate layer positioned between said first and second conductive layers.

22. A face mask according to claim 21, wherein the conductive layers are connected to an electric signal generator such that, in use, a voltage can be applied across said intermediate layer.

23. A face mask according to claim 21, wherein said face mask is a medical or surgical face mask.

24. A face mask according to claim 21, wherein the textile or membrane is configured as a removable cover for said face mask.

25. A method of inactivating microbiological contamination at a locus, the method comprising contacting said locus with a textile or membrane comprising first and second conductive layers and at least one ion conductive or porous intermediate layer positioned between said first and second conductive layers, the textile or membrane further comprising an aqueous liquid on a surface of said textile or membrane or in the pores of a porous intermediate layer, wherein the surface of said textile or membrane contacted with said locus comprises a microbiological contamination-inactivating amount of an inactivating species selected from ozone and reactive oxygen species.
